(19) United States
(12) Patent Application Publication
(10) Pub. No.: US 2015/0238292 A1
(43) Pub. Date: Aug. 27, 2015

(54) DENTAL PROSTHETIC DEVICE WITH REMOLDABLE BASE AND MEDICANT DELIVERY SYSTEM

(71) Applicant: Innovative Products, Inc., Leawood, KS (US)

(72) Inventor: Irwin N. Boe, Leawood, KS (US)

(21) Appl. No.: 14/698,604

(22) Filed: Apr. 28, 2015

Related U.S. Application Data
(63) Continuation-in-part of application No. 13/385,381, filed on Feb. 16, 2012, now Pat. No. 9,017,074.
Application No. 13/385,381 is a continuation-in-part of application No. 13/066,202, filed on Apr. 8, 2011, now abandoned.
Application No. 13/066,202 is a continuation-in-part of application No. 14/250,384, filed on Apr. 11, 2014.

(51) Int. Cl. A61C 13/07; A61C 19/06; A61B 5/00; A61C 13/01

(52) U.S. Cl. CPC A61C 13/0025; A61C 13/01; A61C 19/063; A61B 5/4845

(57) ABSTRACT

A dental prosthetic device having a base constructed of an inexpensive remoldable material to allow for inexpensive and quick fitting and refitting to accommodate changes in dental structure. Harder tooth-like structures are associated with a mounting surface of the base, such as by overmolding the base onto ends of the structures, so that when the device is placed over a dental arch within a user's mouth, the tooth-like structures facilitate biting and chewing in the manner of teeth. A carrier is integrated in or affixed to one or more of the tooth-like structures and carries a treating agent. The treating agent is configured for controlled release over time through reaction with or dissolution by saliva in the mouth or by mechanical abrasion of the agent. The treating agent may be absorbable by mucous membranes in the mouth and may provide medicants, flavorings, aromatics, or the like.

### CROSS-REFERENCE TO RELATED APPLICATIONS

This application is a continuation-in-part of U.S. patent application Ser. No. 13/385,381, filed Feb. 16, 2012, which is a continuation-in-part of U.S. patent application Ser. No. 13/066,202, filed Apr. 8, 2011. This application is also a continuation-in-part of U.S. patent application Ser. No. 14/250,384 filed Apr. 11, 2014. The disclosures of each of these prior applications are hereby incorporated herein in their entirety by reference.

### BACKGROUND

Dental prosthetics, which include partial and complete dentures, can help people in many ways, including improving their appearance and self-esteem as well as their ability to chew food and speak clearly. The usefulness of dental prosthetics is reflected in their at least 2700 year history, during which time they have been made of materials such as bone and wood.

Modern dental prosthetics, especially the base component which contacts the dental arch, are commonly made of acrylic, other hard plastics, or metal. These prosthetics can require several initial appointments over one or two months to construct and achieve a proper fit and appearance as well as periodic adjustments to maintain a proper fit as the shape of users' dental tissues change. For example, following an extraction of many or all teeth, tissue and bone may take many months to heal. During that time, prior art dental prosthetics can suffer problems with support, stability, and retention, and can create sore spots on soft tissue because of changing fit. Furthermore, the underlying bones, particularly the mandibular arch, can continue changing for many years and require periodic refittings, possibly as often as every five to seven years. For these and other reasons, modern dental prosthetics can be prohibitively expensive for some people.

Another known problem is associated with providing efficient and efficacious means for administering a treating agent (i.e., any substance, molecule, element, compound or otherwise active ingredient operable to effect an intended benefit) to a user over an extended period of time. For example, in the medical and dental arts, doctors commonly prescribe treating agents, such as drugs and medicants, to patients for repetitive oral consumption. It is widely appreciated, however, that repetitive oral consumption presents various concerns. Foremost, where self-administered, users, such as the elderly and mentally infirm, often forget or unintentionally fail to adhere to the specified regiment and schedule. This may render the treatment ineffective and in some cases worsen the mal condition. Where manual administration is difficult, as with swallowing large pills/capsules, it is further appreciated that many users become deterred from taking the prescribed agent all together. Further, even where oral consumption is properly performed, inefficiencies, such as the “first pass effect”—the percentage of drug lost to metabolization in the liver, often result in increased costs, waste, and in some cases harmful side effects. It is appreciated that similar human error concerns exist for extended intravenous, and other forms of administration.

Due to these and other problems and disadvantages in the prior art, a need exists for a dental prosthetic device that is less expensive to make, fit, and maintain.

Such a device that can also aid provision of medicants and other agents to users would also be very beneficial.

### SUMMARY

Embodiments of the invention are defined by the claims below, not this summary. A high-level overview of various aspects of the invention is provided here to introduce a selection of concepts that are further described in the Detailed-Description section below. This summary is not intended to identify key features or essential features of the claimed subject matter, nor is it intended to be used in isolation to determine the scope of the claimed subject matter. In brief, this disclosure describes, among other things, a dental prosthetic device with an integrated medicant delivery component.

The dental prosthetic device includes a base component constructed of a soft, inexpensive, remoldable material and having a contact surface and a mounting surface. The contact surface is shaped to fit over a dental arch. A harder component comprising one or more tooth-like structures is associated with the base such that when the dental prosthetic device is placed over the dental arch within a user's mouth, the harder component facilitates biting and chewing in the manner of teeth. The device may further include an intermediate component positioned approximately between the softer base and harder components in order to, for example, better support the tooth-like structures.

At least one of the tooth-like structures includes a medicant delivery component integrated therein or disposed thereon for delivery of an orally administered treating agent, such as a drug, therapeutic agent, medicant, and/or aromatic/flavor producing agent, to a user. The treating agent might also comprise a substance-indicating element that indicates the presence of a substance such as alcohol in the breath or saliva of the user. The component includes a carrier that is attachable to an exterior surface or integrated into a tooth-like structure of the dental prosthetic device. The carrier may provide a partially enclosed interior space that is defined by a plurality of planar slats within which the therapeutic agent may be disposed. Or the carrier may provide a surface on which the treating agent is coated or impregnated. The slats of the carrier and/or the therapeutic agent compound is configured to provide a desired timed or controlled release of the agent while in contact with saliva in the user's mouth.

### DESCRIPTION OF THE DRAWINGS

Illustrative embodiments of the invention are described in detail below with reference to the attached drawing figures, and wherein:

FIG. 1 is a plan view of an upper or maxillary dental prosthetic device depicted in accordance with an embodiment of the invention;

FIG. 2 is a plan view of a lower or mandibular dental prosthetic device depicted in accordance with an embodiment of the invention;

FIG. 3 is a view of a contact surface of the dental prosthetic device of FIG. 1;

FIG. 4 is a view of a contact surface of the dental prosthetic device of FIG. 2;

FIG. 5 is a partially exploded perspective view of the dental prosthetic device of FIG. 2, showing its functional relation to a mandibular arch;

FIG. 6 is a cross-sectional elevational view of the dental prosthetic device of FIG. 1 showing its functional relation to a dental arch in accordance with an embodiment of the invention;

FIG. 7 is a cross-sectional elevation view of a dental prosthetic device showing a first form of an intermediate component depicted in accordance with an embodiment of the invention;

FIG. 8 is a cross-sectional elevation view of a dental prosthetic device showing a second form of an intermediate component depicted in accordance with an embodiment of the invention;

FIG. 9 is a partial cross-sectional perspective view of a dental prosthetic device showing internal support structures in accordance with another embodiment of the invention;

FIG. 10 is a side elevation view of the embodiment of FIG. 9 showing the internal support structures;

FIG. 11 is a bottom view of the embodiment of FIG. 9 showing the internal support structures;

FIG. 12 is a perspective view of a human user with a partial cutaway illustrating an oral cavity and a plurality of teeth or tooth-like structures presented thereby, and further illustrating a medicant-delivery component fixedly bonded to a lower molar, in accordance with an embodiment of the invention;

FIG. 13 is a perspective view of a mandibular dental prosthetic device, and in enlarged caption view, a medicant-delivery component defining an interior space and attached to the exterior lateral surface of a tooth-like structure resembling a molar, in accordance with an embodiment of the invention;

FIG. 13a is a perspective view of the back of the medicant-delivery component shown in FIG. 13;

FIG. 14 is an enlarged perspective view of a button-type medicant-delivery component comprising a coat formed at least in part by an agent, and a base plate having an attachment prong extending from the plate, depicted in accordance with an embodiment of the invention;

FIG. 15 is a perspective view of a mandibular dental prosthetic device with a medicant-delivery component disposed in a tooth-like structure resembling a molar and showing a base of another medicant-delivery component exposed in an interior surface of another tooth-like structure in accordance with an embodiment of the invention;

FIG. 16 is a cross-sectional view of a dental prosthetic device depicting a medicant-delivery component disposed within and extending the width of a tooth-like structure in accordance with an embodiment of the invention;

FIG. 17 is a cross-sectional view of a dental prosthetic device depicting a medicant-delivery component disposed within and extending only partially along the width of a tooth-like structure in accordance with an embodiment of the invention;

FIG. 18 is a perspective view of a maxillary dental prosthetic device with a pad-type medicant-delivery component adhered to the interior surface of two adjacent tooth-like structures depicted in accordance with an embodiment of the invention;

FIG. 19a is a side elevation of a pad-type medicant-delivery component defining a plurality of discrete gaps containing a treating agent, depicted in accordance with an embodiment of the invention;

FIG. 19b is a side elevation of the medicant-delivery component shown in FIG. 19a, partially dissolved, so as to expose internal gaps to the oral cavity;

FIG. 19c is a side elevation of the medicant-delivery component shown in FIG. 19b but more completely dissolved; and

FIG. 20 is a plan view of an upper or maxillary dental prosthetic device with a medicant-delivery pad disposed on a palate thereof depicted in accordance with an embodiment of the invention.

### DETAILED DESCRIPTION

The subject matter of select embodiments of the invention is described with specificity herein to meet statutory requirements. But the description itself is not intended to necessarily limit the scope of claims. Rather, the claimed subject matter might be embodied in other ways to include different components, steps, or combinations thereof similar to the ones described in this document, in conjunction with other present or future technologies. Terms should not be interpreted as implying any particular order among or between various steps herein disclosed unless and except when the order of individual steps is explicitly described.

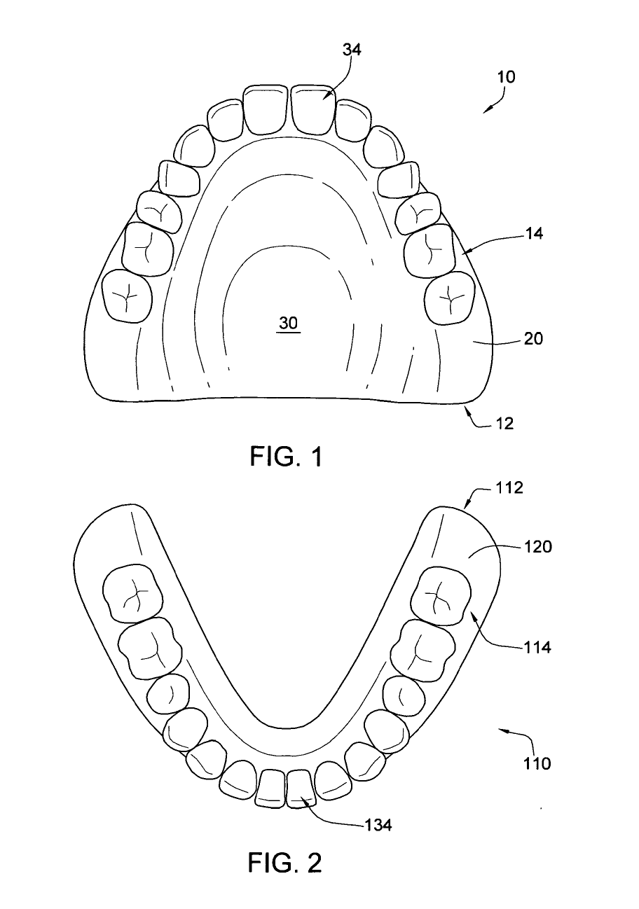

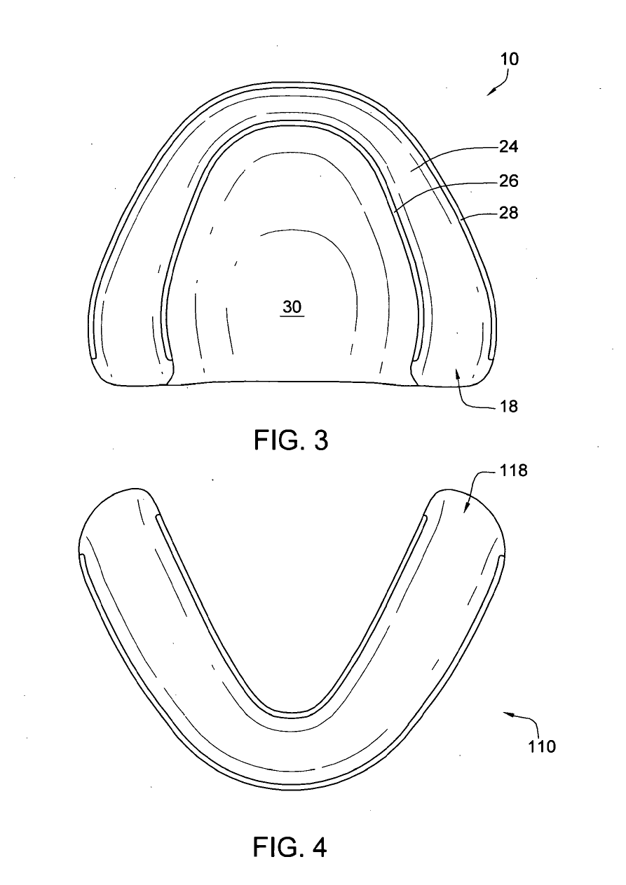

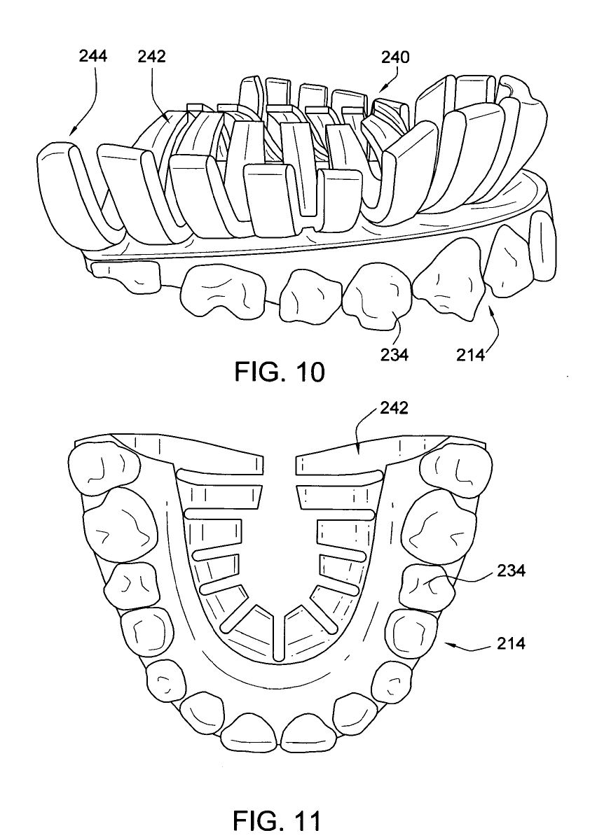

With reference to FIGS. 1-11, a dental prosthetic device 10 is described, shown, and otherwise disclosed in accordance with one or more embodiments of the present invention. FIGS. 1 and 3 show the dental prosthetic device 10 adapted for an upper or maxillary arch, while FIGS. 2 and 4 show the dental prosthetic device 110 adapted for a lower or mandibular arch. The upper and lower adaptations 10, 110 may be substantially identical with regard to materials and method of construction, unless noted otherwise.

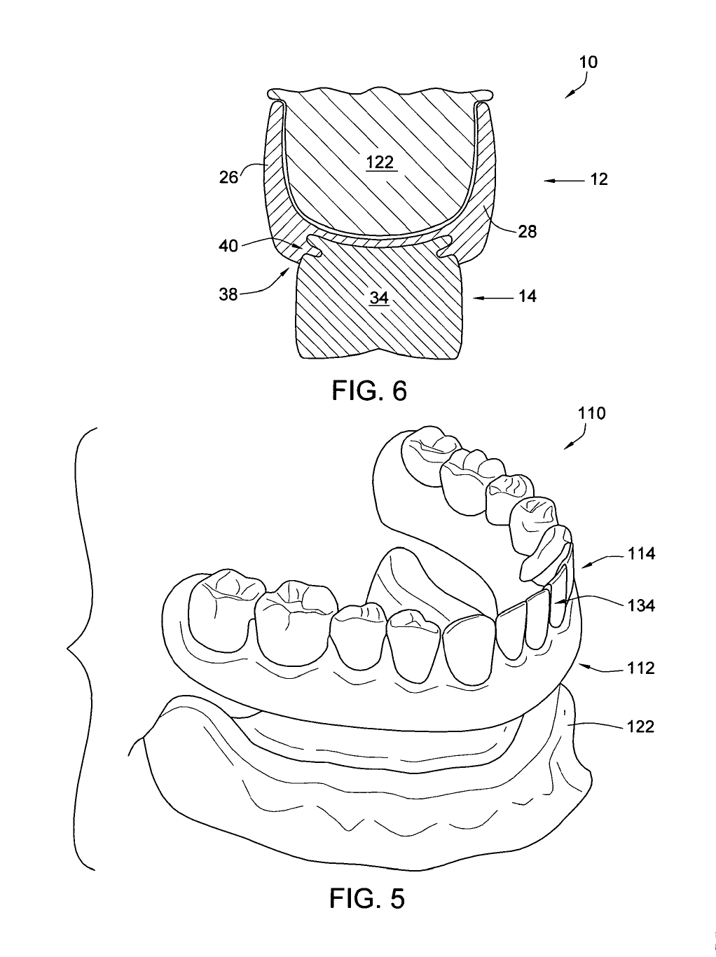

The dental prosthetic device 10, 110 comprises a softer base component 12, 112 and a harder component 14, 114. The softer base component 12, 112 includes a contact surface 18, 118 and a mounting surface 20, 120. Referring also to FIGS. 5 and 6, the contact surface 18, 118 is generally shaped to fit over the dental arch 122. More specifically, the contact surface 18, 118 may be generally channel-shaped with a groove 24 running between inner and outer sidewalls 26, 28, so that the dental arch 122 is at least partially received within the groove 24. The maxillary version of the device 10 may include a center portion 30, as shown in FIG. 1, or may exclude the center portion and be substantially U-shaped like the mandibular version 110 shown in FIG. 2.

The softer base component 12, 112 may incorporate a remoldable elastomeric or thermoplastic material, such as, for example, ethylene vinyl acetate, which, when activated, such as by being warmed at ambient pressure to a relatively low temperature, such as, for example, approximately 105° C. or lower, between 85° C. and 105° C., between 50° C. and 85° C., or lower than 50° C., conforms more closely to the dental arch 122. The terms “about” or “approximately” as used herein denote deviations from the exact value by +/−10%, preferably by +/−5% and/or deviations in the form of changes that are insignificant to the function. More specifically, a relatively close fit can be achieved by warming or otherwise activating the softer base component 12, 112 and placing the contact surface 18, 118 over the dental arch 122 so that the material of the softer base component more closely conforms to the shape of the dental arch 122. Furthermore, the softer base component 18, 118 can be rewarmed or reactivated whenever necessary or desirable to remold it to the changing shape of the dental arch 122. The softer base component 12, 112 may be colored to, for example, resemble gum tissue appropriate to the user's race.

The harder component 14, 114 includes one or more tooth-like structures 34, 134 associated with the mounting surface 20, 120 of the softer base component 12, 112 such that, when the dental prosthetic device 10, 110 is placed within a user's mouth in the manner of a denture, the harder component 14, 114 facilitates biting, chewing, and speaking in the manner of natural teeth. The harder component 14, 114 may incorporate a molded acrylic material. The one or more tooth-like structures 34, 134 may be affixed to or, as shown in FIG. 6, embedded in the softer base component 12, 112, such as, for example, by overmolding the softer base component 12, 112 onto ends 38 of the one or more tooth-like structures 34, 134. The ends 38 of the one or more tooth-like structures 34, 134 may be provided with grooves, holes, or other features 40 to better maintain their relationship with the softer base component 12, 112. The one or more tooth-like structures 34, 134 may be shaped and colored to resemble natural teeth, or may be shaped and/or colored in substantially any manner of functionally or aesthetically desirable ways.

The dental prosthetic device 10, 110 may be produced in a limited number of sizes, such as, for example, small, medium, and large, which provides a first order of fit. Heating or otherwise activating the softer base component 12, 112 and molding it to the user's particular dental arch provides a second order of fit. As discussed, the user can thereafter reheat or otherwise reactivate the soft base component 12, 112 to refit the device 10, 110 whenever necessary due, for example, to changing tissue shape.

In one exemplary embodiment incorporating certain of the aforementioned features, the dental prosthetic device 10, 110 may comprise the softer base component 12, 112 being constructed of a remoldable material and having the contact surface 18, 118 and the mounting surface 20, 120, with the contact surface 18, 118 being shaped to fit over the dental arch 122. The softer base component 12, 112 may be colored to resemble gum tissue. The harder component 14, 114 may comprise the one or more tooth-like structures 34, 134 partially embedded in the softer base component 12, 112 and extending beyond the mounting surface 20, 120 of the softer base component 12, 112. The one or more tooth-like structures 34, 134 are shaped and colored to resemble natural teeth. As such, when the dental prosthetic device 10, 110 is placed within a user's mouth, the harder component 14, 114 facilitates biting and chewing in the manner of natural teeth.

In another exemplary embodiment incorporating certain of the aforementioned features, the dental prosthetic device 10, 110 may comprise the softer base component being constructed of a remoldable thermoplastic material which, when warmed, conforms more closely to the dental arch 122. The softer base component 12, 112 includes the contact surface 18, 118 and the mounting surface 20, 120, with the contact surface 18, 118 being shaped to fit over the dental arch 122, and the softer base component 12, 112 being colored to resemble gum tissue. The harder component 14, 114 includes one or more tooth-like structures 34, 134 constructed of a molded acrylic material embedded in the softer base component 12, 112 and extending beyond the mounting surface 20, 120 of the softer base component 12, 112. The one or more tooth-like structures are shaped and colored to resemble natural teeth such that when the dental prosthetic device 10, 110 is placed within a user's mouth, the harder component 14, 114 facilitates biting and chewing in the manner of natural teeth.

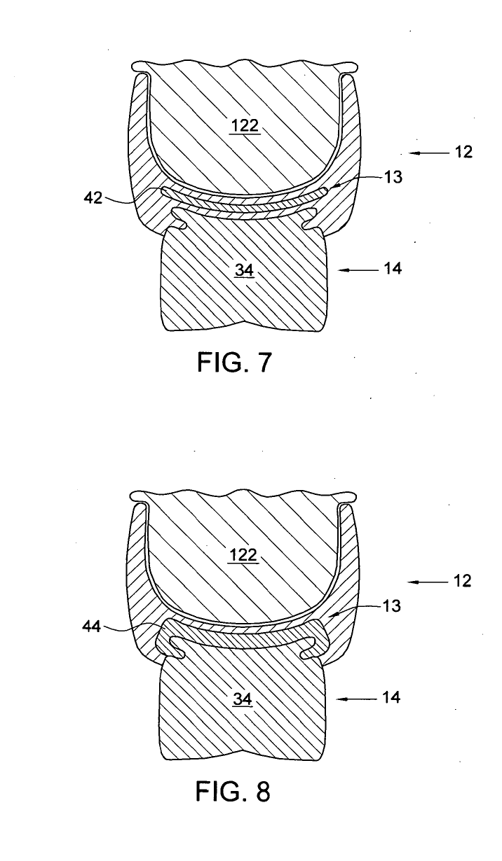

Referring also to FIGS. 7 and 8, the dental prosthetic device 10, 110 may further comprise an intermediate component 13 positioned approximately between the softer base component 12, 112 and the harder component 14, 114. The intermediate component 13 may be constructed of a harder material than the softer base component 12, 112 in order, for example, to provide greater durability or wear-resistance or to better maintain or secure the harder component 14, 114 relative to the softer base component 12, 112. Thus, the intermediate component 13 may be constructed of, for example, a harder version of the material of the softer base component 12, 112, a material having a hardness which is intermediate to that of the softer base component 12, 112 and the harder component 14, 114, or a hardness which is as hard or harder than the hardness of the harder component 14, 114. Furthermore, the intermediate component 13 may take a first form 42 in which it touches or does not touch (as shown in FIG. 7) one or both of the other components 12, 112, 14, 114, or may take a second form 44 in which it engages one or both of the other components 12, 112, 14, 114 (as shown in FIG. 8).

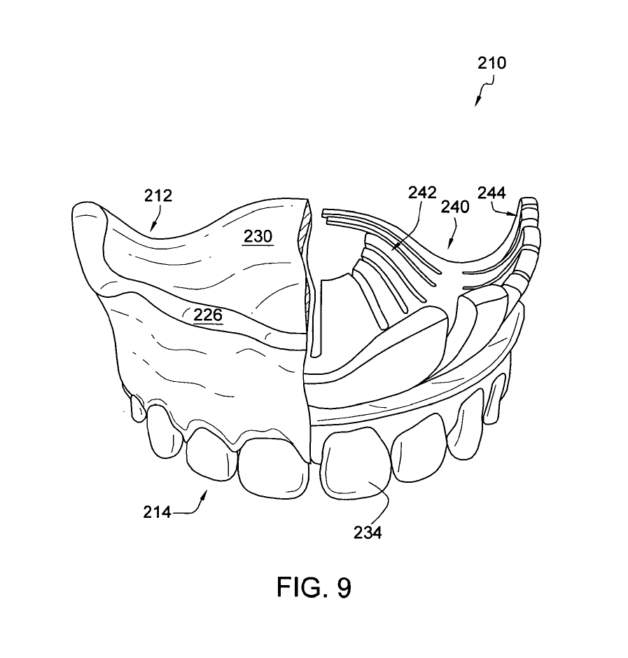

With reference to FIGS. 9, 10, and 11, a second embodiment of the dental prosthetic device 210 is now described, shown, and otherwise disclosed. The second embodiment may be substantially similar or identical to the first embodiment except as follows. Although the device 210 is shown only adapted for the upper or maxillary arch, it will be appreciated that the following may apply also to the device as adapted for the lower or mandibular arch.

The dental prosthetic device 210 comprises the softer base component 212 and the harder component 214 as discussed above. The harder component 214 includes the one or more tooth-like structures 234 and one or more internal support structures 240 that are embedded within the softer base component 212 and may function to support the tooth-like structures 234 in relation to the softer base component 212 or to achieve or maintain a better fit within the user's mouth.

More specifically, as seen in FIG. 9, the internal support structures 240 may take the form of ribs or fingers of material extending from the tooth-like structures 234 into the center portion 230 and/or the sidewall(s) 226 (depicted in FIG. 6 as reference numerals 26 and 28) of the device 210 and covered by, e.g., overmolded with the softer base component 212, thereby providing increased support while still benefiting from the remoldability of the softer base component 212. The center portion internal support structures 242 extend into the center portion of the maxillary version of the device 210, and may be sufficiently flexible to substantially conform to differently shaped, e.g., rounder or flatter palates. The sidewall internal support structures 244 extend into the inner and/or outer sidewalls 26, 28 of either the maxillary or the mandibular versions of the device 210 and may be sufficiently flexible to substantially conform to differently shaped dental arches. In both cases, the internal support structures 240 may extend at least half-way, between half-way and three-quarters of the way, or substantially all of the way (possibly even joining at the apex of the center portion) into the center portion 230 and at least half-way, between half-way and three-quarters of the way, or substantially all of the way into the sidewall(s) 226 for better support. In various implementations, there may a single internal support structure, one internal support structure shared by several of the tooth-like structures, or one internal support structure for each of the tooth-like structures.

The tooth-like structures 234 may be made of acrylic or of a more flexible or softer material such as polyethylene or polycarbonate. The latter softer materials may wear faster than the former harder material, but it is anticipated that the relatively low cost of the device 210 (as compared to traditional dentures) will allow for more frequent replacement. The internal support structures 240 may be constructed of the same material, a more flexible or softer version of the material, or a different material than the tooth-like structures 234. In particular, at least a flexible or softer version of the material of the tooth-like structure may be desirable to minimize the risk that the internal support structures 240 may tear through the softer base component 212 in which they are embedded and come into direct contact with the user's oral tissues. Relatedly, the tooth-like structures 234 and the internal support structures 240 may be molded or otherwise formed simultaneously or at different times or as a single piece or as multiple pieces.

In one implementation, for example, the tooth-like structures 234 and the internal support structures 240 are molded simultaneously as a single piece but the tooth-like structures 234 are constructed of a harder version of a material (e.g., acrylic) and the internal support structures 240 are constructed of a softer or more flexible version of the material. This may be accomplished, for example, by creating a continuous gradient of one or more additive materials in the mold such that little or none of the additive material(s) is present in the portion of the mold corresponding to the tooth-like structures 234 and more of the additive material(s) is present in the portion of the mold corresponding to the internal support structures 240, resulting in a gradient of hardness of other characteristic(s) from the harder and/or relatively inflexible tooth-like structures 234 to the softer and/or relatively flexible internal support structures 240. Such a gradient may be created, for example, through careful positioning or orientation of the mold, the use of gravity to separate heavier or denser material from lighter or less dense material, or by creating the device 210 from the tooth-like structures 234 to the internal support structures 240 and slowly introducing more of the additive material into the latter once the former has partially cured or will otherwise no longer accept the additional additive material.

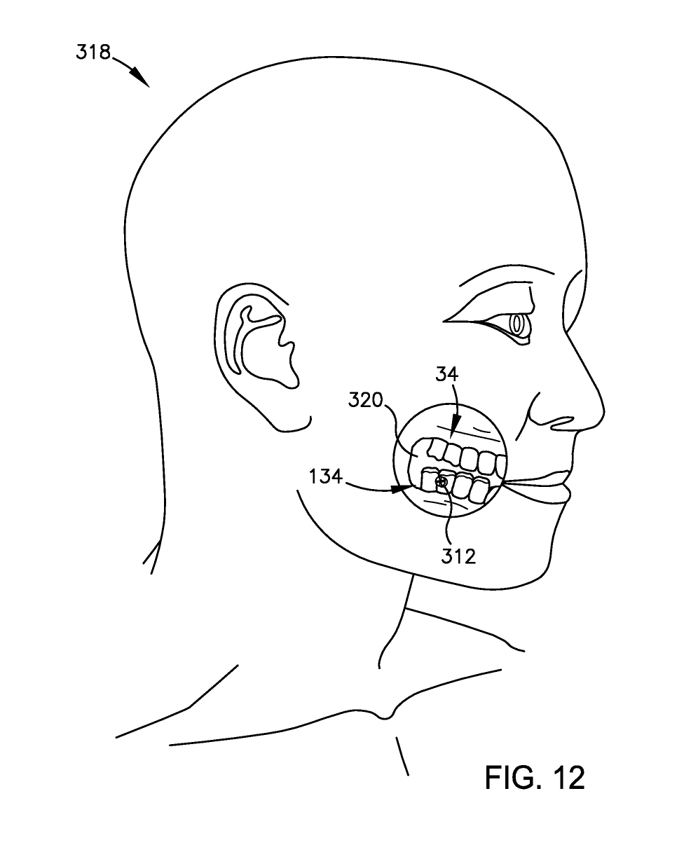

With reference now to FIGS. 12-15, in one embodiment, the dental prosthetic device 10, 110 may further comprise a medicant-delivery component 312 for orally administering a treating agent 314 or a compound 316 that includes the agent 314 to a user 318 via an oral cavity 320 of the user over an extended period. As used herein the term “treating agent” comprises any substance, molecule, element, or otherwise active ingredient operable to affect an intended benefit within the user 318 through physical or chemical engagement therewith. Among other things, the agent 314 may be a therapeutic agent, medicant, drug, aromatic/flavor producing agent, a combination of the above, and/or the like. Exemplary drugs and medicants may further include alkylating, anti-metabolite, analgesic, or anti-anxiety agents. For example, in a dental setting, the agent 314 may present a plurality of monocycline microspheres (e.g., monocycline HCl) that may be used to fight periodontitis. In another embodiment, the “treating agent” comprises a component, compound, substance, or device that reacts with or otherwise detects the presence of another chemical or substance in the oral cavity 320 and provides an indication thereof, e.g. the treating agent may indicate the presence of an alcohol in the user's breath or saliva by reacting therewith to cause a visible color change in the treating agent.

The treating agent 314 may compose a compound 316 operable to effect additional functionality (e.g., promote curing, control the release of the agent, or modify a cavity condition, so as to facilitate delivery/absorption, etc.). For example, the compound 316 may further include an effervescent couple used to enhance drug penetration/absorption across the buccal (inside cheek), sublingual (under the tongue), and gingival (between the lips and gum) mucosae. The preferred effervescent couple evolves gas by means of a chemical reaction triggered by exposure to saliva in the mouth. For example, a soluble acid source, such as citric acid, may be caused to react with a source of carbon dioxide that is mostly basic, such as an alkaline carbonate or bicarbonate, so as to produce carbon dioxide gas. Alternatively, a pH adjusting substance may be included in the compound 316, as it is appreciated that pH levels can influence the relative concentrations of ionized and un-ionized drug, which in turn, affects the dissolution of the drug in the saliva and absorption across the oral mucosa.

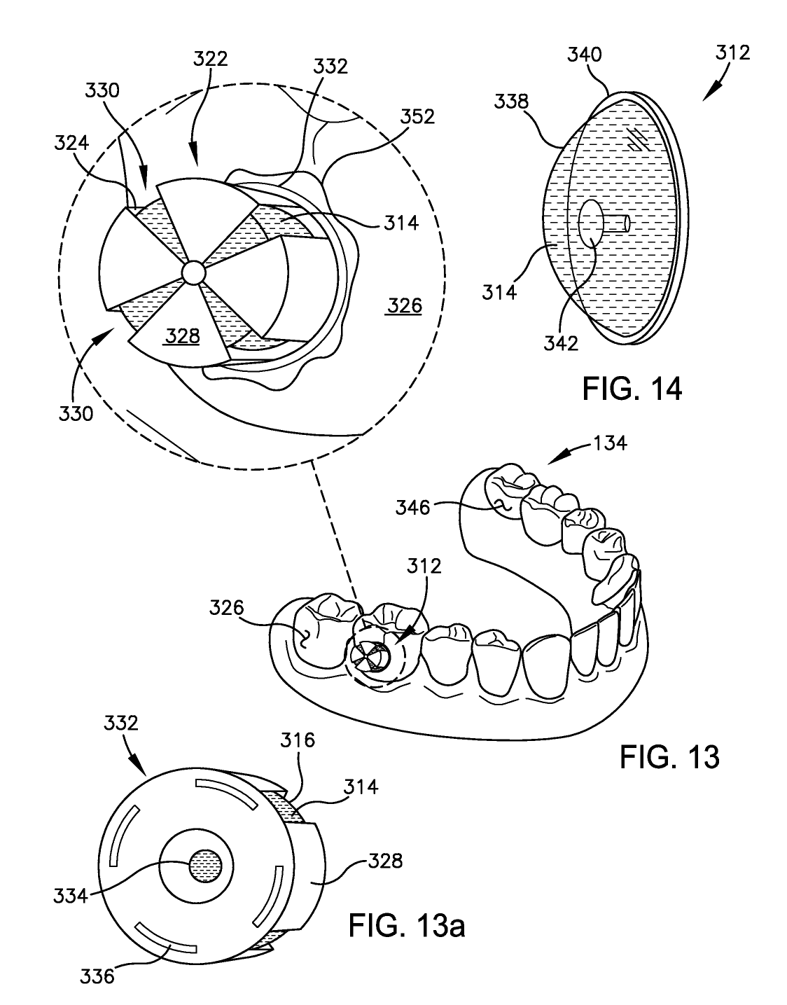

In an embodiment, the agent 314 and/or compound 316 is retained by a carrier 322 intermediately affixed to the tooth-like structure 34/134 and operable to provide a controlled release of the agent 314. As shown in FIG. 13, for example, the medicant-delivery component 312 may include a reservoir-type carrier 322, wherein the carrier 322 defines a partially enclosed interior space 324 and is attachable to the exterior lateral surface 326 of a rear molar tooth-like structure 34/134. Although embodiments of the invention are described herein with respect to the carrier 322 being associated with a tooth-like structure 34/134 resembling a molar, such is not intended to limit association of the carrier 322 with any particular tooth-like structure 34/134, e.g. the carrier 322 may be associated with tooth-like structures 34/134 resembling molars, incisors, bicuspids, canines, or the like. The carrier 322 may also be at least partially disposed in or associated with the base component 12, 112 and/or the internal support structures 240 of the prosthetic device 10, 110.

The carrier 322 comprises, and the space 324 is defined by, a plurality of polymeric planar slats 328 radially emanating from a disk base 332. The base 332 is indiscriminately circular in the illustrated embodiment, and defines an insertion hole 334 for receiving the agent 314. A plurality of radially open sectors 336 may be defined adjacent the perimeter of the base 332 for added access to and from the space 324 (FIG. 13a). The planar slats 328 converge at the apex of the carrier 322 opposite the base 332, and are preferably tapered to facilitate convergence. The agent 314 is disposed (e.g., injected or inserted) within the space 324, so that the carrier 322 encapsulates the agent 314. In their final configuration, the planar slats 328 define intermediate gaps 330 configured to allow fluid seepage to and from the space 324.

As such, once in place, saliva is allowed to flow into the space 324 and interact with the agent 314/compound 316, so as to release the agent 314 in a controlled manner. That is to say, the agent 314/compound 316 may be configured to chemically react with, or be slowly dissolved by saliva at a rate configured to affect a desired time release. For example, the compound 316 may present or the agent 314 may be otherwise retained by hydrolysable bonds that break when exposed to the water content of saliva. To release the agent 314 at a different rate, the constituency of the compound 316 may be changed such that the bonds become hydrolyzed at a different rate. Alternatively, the agent 314 may compose a gel, or other high viscosity fluid operable to affect the desired time release, through shearing due to gravity.

In one embodiment, the agent 314 is disposed within a porous medium, such as a polymer matrix configured to allow the agent 314 to escape or diffuse therefrom at a controlled rate. The porous medium may be installed inside the carrier 322 within the space 324 behind the planar slats 328, or the planar slats 328 might be omitted because they are not necessary to control the diffusion rate of the agent 314. For example, the porous medium comprising the carrier 322 in FIG. 19a might be comprised of a non-dissolving and/or non-eroding material with one or more interconnecting pores through which the agent 314 may be released at a controlled rate.

The gaps 330 and planar slats 328 may be adjustable, so as to vary the rate of seepage and therefore time release, for example, by pushing down on the apex to cause resistively bendable planar slats 328 to spread radially. Alternatively, the component 312 may further include a manually shiftable outer cover (not shown) that shifts between exposed and closed positions, such that the agent 314 is exposed to the oral cavity 320 and released only when the cover is in the exposed position.

In another embodiment, the agent 314 or compound 316 may be coated onto the carrier 322 (FIG. 14). More particularly, the component 312 may present a hard coat 338 formed at least in part by agent 314 over-molded upon a solid base 340 or directly on the surface of the tooth-like structure 34/134, wherein the term “coat” is not limited to thin superjacent layers, but includes three-dimensional molds of material. The underlying base 340 may be of any suitable shape or form, so long as it enables attachment to the tooth-like structure 34/134 and provides enough surface area for receiving the coat 338. In the exemplary configuration shown in FIG. 14, an attachment prong or stem 342 extends from the base 340, and provides increased surface area and structure for retaining the coat 338.

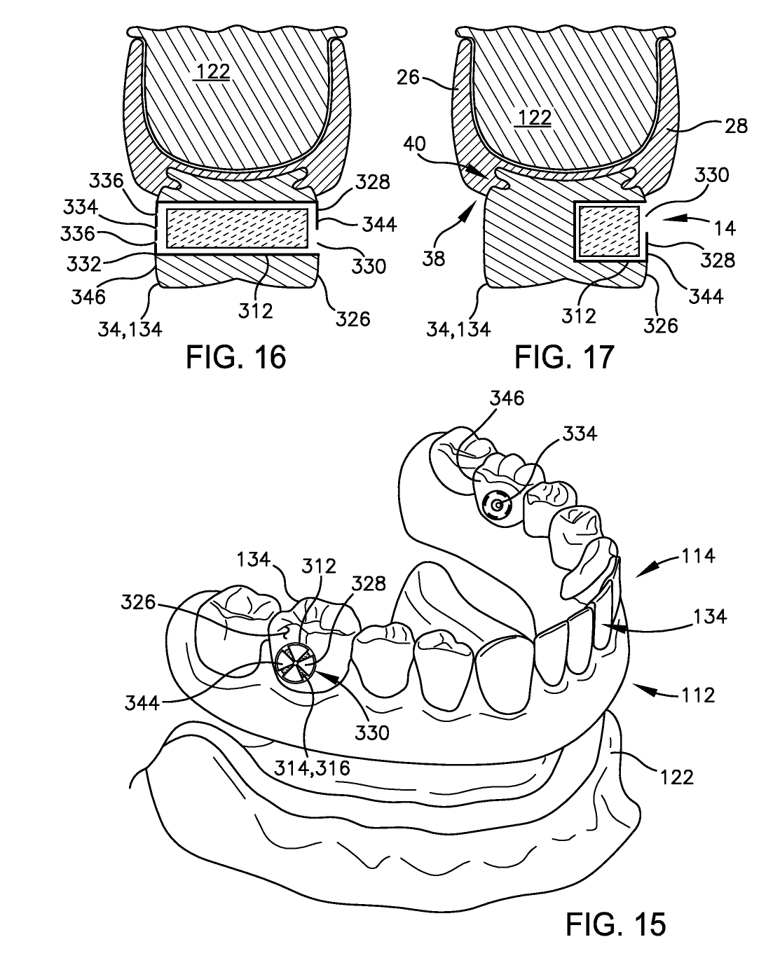

In another embodiment depicted in FIGS. 15-17, the medicant-delivery component 312 may be integrated into one or more of the tooth-like structures 34/134 to present an exposed face 344 that is substantially flush with one or more surfaces of the tooth-like structure 34/134. The face 344 is preferably positioned on the exterior lateral surface 326 of the tooth-like structure 34/134 but may additionally or alternatively provided on the interior lateral face 346 or on another surface of the tooth-like structure 34/134. In another embodiment, the medicant-delivery component 312 may present an exposed face 344 that is at least partially within the softer base component 12/112.

The medicant-delivery component 312 may extend across the full width of the tooth-like structure 34/134 (FIG. 16), e.g. between the exterior and interior lateral surfaces 326, 346, or may extend only partially across the width thereof (FIG. 17). As such, the exposed face 344 and the slats 328 and gaps 330 presented thereby may be provided on either or both of the exterior and interior lateral surfaces 326, 346 of the tooth-like structure.

The medicant-delivery component 312 may be removable from the tooth-like structure 34/134 to enable insertion of the agent 314 or compound 316 therein. For example, the medicant-delivery component 312 may be slideable in an axial direction within a bore in the tooth-like structure 34/134 in which the component 312 is disposed. Or the component 312 may remain integral with the tooth-like structure 34/134 during installation of the agent 314/compound 316. In one embodiment, the tooth-like structure 34/134 is removable from the dental prosthetic device 10, 110 to aid installation of the agent 314/compound 316. As depicted in FIG. 15, where the medicant-delivery component 312 extends across the width of the tooth-like structure 34/134, the insertion hole 334 and/or open sectors 336 may be provided on one of the exterior or interior lateral surfaces 326, 346 of the tooth-like structure 34/134 to enable insertion of the agent 314 or compound 316 into the component 312.

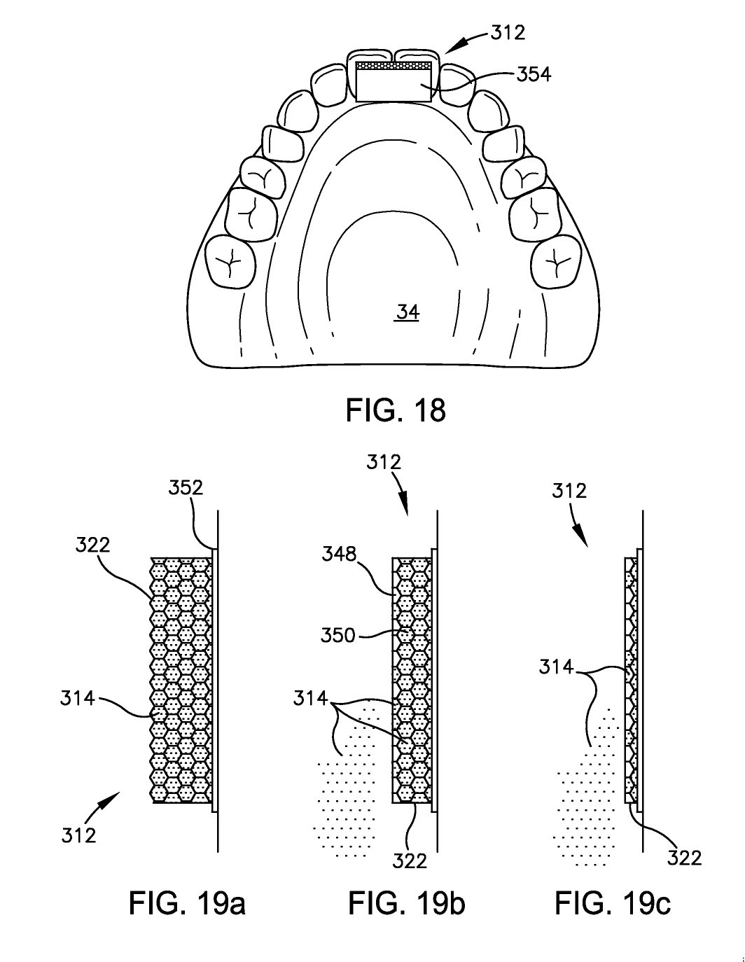

With reference now to FIGS. 18 and 19a-c, in another embodiment, the carrier 322 may be formed interstitially by a matrix, honeycomb, or the like, which defines an interconnected labyrinth or plurality of discrete gaps 348 (FIGS. 19a-c). In this configuration, the agent 314 and/or compound 316 is stored within the gaps 348, and gradually released over the period. For example, the carrier 322 may be formed of a polymer matrix, wherein the agent 314 is combined within the polymer as an ingredient prior to fabrication. Once molded and implanted, the agent 314 diffuses from the carrier 322 gradually. In one example, two polymers may be mixed with a macromolecular drug, which diffuses out of the component 312 in a controlled fashion, gradually escaping through gaps 348 in the polymer matrix over a ninety-day period. It is appreciated that the macromolecular drug is held in place by intermolecular “hydrophobic” interactions that facilitate its slow diffusion through the matrix.

The matrix material may be dissolvable over the period, so as to gradually expose the interior gaps 350 to the releasing conditions of the cavity 320 (FIGS. 19a-c). As a result, the agent 314 stored within the internal gaps 350 are incrementally released as layers of the matrix wear off, and may be a conventional liquid or solid that is instantaneously released once exposed. Suitable dissolvable material must be digestible without harm by the user 318, and may include starches and cellulose material.

Especially where the agent 314 presents a prescription strength drug or medicant, the component 312 is intended for insertion or attachment (and removal where necessary) by a trained healthcare provider. In a dental setting, for example, the carrier 322 may be bonded to at least one tooth-like structure 34/134 by applying a quantity of a preloaded dental composite material 352 (e.g., a glass ionomer) intermediate the carrier 322 and tooth-like structure 34/134 (FIGS. 13 and 19a-c), and curing the material 352 with a non-ultraviolet light source (not shown). Again, it is appreciated that the hold strength of the material 352 is such that the component 312 does not become dislodged through ordinary usage of the mouth. Thus, a preferred method further includes manually breaking the bond through instrumentation and/or exposing the material 352 to a dissolving agent after use.

It is certainly within the ambit of the present invention, however, for the user 318 to self-apply an over-the-counter component 312, particularly where the agent 314 is aromatic or flavor producing. For example, the component 312 may comprise a pad-type implantation component 354 formed of a dissolvable matrix impregnated with an aromatic and/or flavor producing agent may be adhered to an interior lateral surface 346 of the tooth-like structures 34/134, so as to be adjacent the tongue region best suited to taste the flavor. In FIGS. 18 and 19a-c, a pad-type component 354 comprising a sweet flavor producing agent is adhered to the interior surface 346 of the two front-most tooth-like structures 34, so as to engage the tip of the user's tongue. Interior disposition of aromatic agents 314 may further offer a discrete solution to halitosis and other mal conditions.

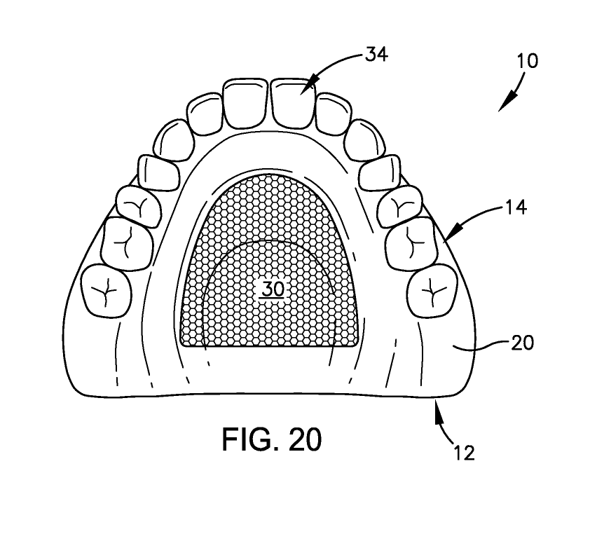

With additional reference to FIG. 20, in another embodiment, the component 312 comprises a pad-type component 356 configured for application to or integration with a palate portion 358 of the maxillary dental prosthetic device 10, e.g. the component 356 may be attached to or integrated with a portion of the device 10 that lies between the interior lateral faces 346 of the tooth-like structures 34 or within the maxillary arch. The component 356 may be installable and replaceable by the user or by medical personnel and may be configured to release the treating agent 314 therein by dissolution or erosion as described herein.

In operation, the medicant-delivery component 312 is affixed relative to one or more of the tooth-like structures 34/134 of the maxillary or mandibular dental prosthetic devices 10/110. The component 312 stays in place autonomously, as opposed to being held in place by clenching the jaw or devices 10/110, which distinguishes the invention from trays, mouthpieces, and the like. The component 312 functions to deliver the agent 314 to a remainder portion of the user 318 so as to be further distinguishable from whitening strips, and the like, which deliver an agent directly to the teeth. It is appreciated that the bond or holding strength between the component 312 and engaged tooth-like structure 34/134 is such that the component 312 does not dislodge under stresses caused during normal operation of the mouth (e.g., tooth brushing, consumption of food and beverages, speaking, etc.). Moreover, the component 312 is configured such that fluid interaction with anticipatory elements and ingredients commonly introduced within the mouth, including fluoride in toothpaste, does not substantially impact the rate of time release or otherwise alter the component 312.

As shown in FIGS. 12-19c, the invention is employed within the oral cavity 320 by affixing the agent 314 relative to at least one of the tooth-like structures 34/134, and more specifically, by directly or indirectly attaching the component 312 to a lateral surface 326, 346 of the tooth-like structure 34/134 or integrating the component 312 with one or more of the tooth-like structures 34/134. By integrating the component 312 or by limiting engagement to the lateral surfaces 326, 346, the component 312 does not interfere with the normal function of the tooth-like structure 34/134. Further, when affixed to the exterior of a tooth-like structure 34/134, the component 312 is preferably attached to the exterior lateral surface 326, so as to minimize interaction with the tongue. The component 312 is configured such that the agent 314 is time releasable (e.g., gradually, incrementally, etc.) within the oral cavity 320 over a predetermined extended period of time, preferably not less than one hour, more preferably not less than one day, and most preferably not less than one month, depending upon the application. For example, it is appreciated that relief from temporary mal conditions, such as allergies or headaches, may be accomplished through the gradual release of a suitable agent over a 12 to 24 hour period.

As a result of releasing the agent 314 over the period, the agent 314 is delivered systemically or locally to a remainder portion (e.g., the bloodstream, gums, etc.) of the user 318. Once depleted over the period, the component 312 may be removed and replaced, or refilled in place. An efficient method of delivering a drug or medicant agent 314 to the user 318 is through the mucous membrane lining within the cavity 320. The invention functions to that end by releasing the agent 314, so as to be absorbed across the lining.

In another embodiment, the treating agent 314 is configured not for release and treatment of the user, but rather as an indicator of substances that may be present in the oral cavity 320 of the user. For example, the agent 314 may configured to react with and/or indicate the presence of alcohol, drugs, or other substances in the saliva or breath of the user. In one embodiment, the agent 314 provides an indication that the blood-alcohol content of the user is above at least a minimum level, such as by reacting with ethanol concentrations present in the breath or saliva of the user. When in the presence of such a substance, the treating agent 314 may provide a visible color change that can be viewed by a user or third party. The color change may be permanent so as to provide a record of exposure or may be reversible to allow continued or repeated use of the agent 314 and/or component 312.

In one embodiment, the component 312 includes one or more electronic sensors configured to detect the presence of alcohol, drugs, or other substances in the saliva or breath of the user. The component 312 may also be configured to communicate wirelessly or through a wired connection an indication of conditions sensed in the oral cavity. For example, the component 312 might communicate an indication to a receiving device when a blood alcohol level above a predetermined level is detected. In such an instance, the component 312 may be configured as an active or passive RFID (radio frequency identification) device or may include appropriate electronic systems to enable communications such as via BLUETOOTH, NFC (near field communications), or other wireless communication systems. The receiving device may include any device configured to receive wireless communications including, for example, a dedicated reader, a smart phone, or a desktop, laptop, or tablet computer, among a variety of other devices. The component 312 may alternatively or additionally store data in a memory that is indicative of the sensed conditions within the oral cavity 320. The stored data may be downloaded from the memory by communicatively coupling the component 312 to a computing device through a wired connection when the prosthetic device 10/110 is removed from the oral cavity 320.

Many different arrangements of the various components depicted, as well as components not shown, are possible without departing from the scope of the claims below. Embodiments of the technology have been described with the intent to be illustrative rather than restrictive. Alternative embodiments will become apparent to readers of this disclosure after and because of reading it. Alternative means of implementing the aforementioned can be completed without departing from the scope of the claims below. Identification of structures as being configured to perform a particular function in this disclosure and in the claims below is intended to be inclusive of structures and arrangements or designs thereof that are within the scope of this disclosure and readily identifiable by one of skill in the art and that can perform the particular function in a similar way. Certain features and sub-combinations are of utility and may be employed without reference to other features and sub-combinations and are contemplated within the scope of the claims.

What is claimed is:

1. A dental prosthetic device comprising:
a base component constructed of a remoldable material and having a contact surface and a mounting surface, the contact surface being shaped to fit over a dental arch and the base component being colored to resemble gum tissue;
a second component comprising one or more tooth-like structures at least partially embedded in and extending beyond the mounting surface of the base component, the one or more tooth-like structures being shaped and colored to resemble teeth, and when placed within a user's mouth, the second component facilitates biting and chewing in the manner of teeth;
a carrier associated with at least one of the tooth-like structures of the second component; and
a treating agent carried by the carrier.

2. The dental prosthetic device of claim 1, wherein the carrier is integrated at least partially into the tooth-like structure.

3. The dental prosthetic device of claim 2, wherein the carrier is exposed on one or both of an exterior lateral surface and an interior lateral surface of the tooth-like structure.

4. The dental prosthetic device of claim 3, wherein the carrier extends between the exterior lateral surface and the interior lateral surface of the tooth-like structure.

5. The dental prosthetic device of claim 1, wherein the carrier includes a plurality of slats that at least partially enclose an interior space of the carrier in which the treating agent is disposed, the slats being configured to form gaps therebetween, the gaps allowing liquid to flow into and out of the interior space and into contact with the treating agent.

6. The dental prosthetic device of claim 1, wherein the carrier is coupled to an exterior surface of the tooth-like structure.

7. The dental prosthetic device of claim 1, wherein the treating agent is coated on the carrier.

8. The dental prosthetic device of claim of claim 1, wherein the remoldable material comprises a thermoplastic material.

9. The dental prosthetic device of claim 1, wherein the second component is constructed of a molded acrylic material.

10. The dental prosthetic device of claim 1, wherein the base component is overmolded onto ends of the one or more tooth-like structures.

11. The dental prosthetic device of claim 1, wherein the treating agent comprises one or more of a therapeutic agent, a medicant, an aromatic agent, and a flavor agent.

12. The dental prosthetic device of claim 1, wherein the treating agent provides an indication of the presence of a substance in at least one of the breath and saliva of the user.

13. The dental prosthetic device of claim 1, wherein the treating agent is at least partially dissolvable by saliva and is at least partially absorbable by a mucous membrane lining within the user's mouth.

14. The dental prosthetic device of claim 1, wherein the treating agent includes an ingredient operable to adjust the pH within the user's mouth.

15. The dental prosthetic device of claim 1, wherein the carrier is at least partially formed by a polymer matrix and the treating agent is embedded within the matrix, and wherein the treating agent is diffusible from the matrix over time.

16. The dental prosthetic device of claim 1, wherein the carrier is formed at least partially from a dissolvable matrix and the treating agent is embedded within the matrix.

17. The dental prosthetic device of claim 1, wherein the carrier is at least partially embedded in the base component.

18. A dental prosthetic device comprising:
a base component constructed of a remoldable material and having a contact surface and a mounting surface, with the contact surface being shaped to fit over a dental arch, and the base component being colored to resemble gum tissue;
a second component comprising one or more tooth-like structures at least partially embedded in and extending beyond the mounting surface of the base component, the one or more tooth-like structures being shaped and colored to resemble teeth, and when placed within a user's mouth, the second component facilitates biting and chewing in the manner of teeth;
a carrier disposed at least partially within at least one of the tooth-like structures of the second component, the carrier including a face that is presented on an exterior and interior lateral surface of the tooth-like structure, the face including one or more slats that at least partially enclose an interior space of the carrier, the one or more slats defining a plurality of gaps through which liquid may flow into the interior space; and
a treating agent disposed within the interior space of the carrier.

19. The dental prosthetic device of claim 18, wherein the carrier extends between the exterior and the interior lateral surfaces of the tooth-like structure, and wherein the carrier further includes a base positioned opposite the face, the base including an aperture through which the treating agent may be installed into the interior space of the carrier.

20. The dental prosthetic device of claim 18, wherein the carrier is removable from the tooth-like structure.

21. The dental prosthetic device of claim 18, wherein the carrier comprises a hollow body that is installed in a bore in the tooth-like structure.

22. A dental prosthetic device comprising:
a base component constructed of a remoldable material and having a contact surface and a mounting surface, with the contact surface being shaped to fit over a dental arch, and the base component being colored to resemble gum tissue;
a second component comprising one or more tooth-like structures at least partially embedded in and extending beyond the mounting surface of the base component, the one or more tooth-like structures being shaped and colored to resemble teeth, and when placed within a user's mouth, the second component facilitates biting and chewing in the manner of teeth;
a carrier associated with at least one of the base component and the second component; and
a treating agent carried by the carrier.

23. The dental prosthetic device of claim 22, wherein the second component further comprises:
an internal support structure connected to and extending from each of the one or more tooth-like structures, the internal support structures being embedded within a central portion of the base component, the central portion being shaped to fit against an oral palate.

24. The dental prosthetic device of claim 22, wherein the treating agent comprises an indicator that indicates the presence of a substance in the breath or saliva of the user.

25. The dental prosthetic device of claim 24, wherein the treating agent indicates at least a minimum blood alcohol content in the user's breath or saliva.
